SEQUENCE LISTING (62 sequences)

SEQ ID NO: 1 - RNA, 411 nt, synthetic construct
  misc_feature 1..411: note = modified sequence of DENV3 capsid
  source 1..411: mol_type = other RNA
gtaatacgactcactatagggagttgttagtctacgtggaccgacaagaacagtttcgactcggaagcttgcttaacgtagtgctgacagttttttattagagagcagatctctgatgaacaaccaacggaaaaagacgggaaaaccgtctatcaatatgctgaaacgcgtgagaaaccgtgtgtcaactggatcacagttggcgaagagattctcaaaaggattgctgaacggccaaggaccaatgaaattggttatggcgttcatagctttcctcagatttctagccattccaccaacagcaggagtcttggctagatggggaaccttcaagaagtcgggggccattaaggtcctgaaaggcttcaagaaggagatctcaaacatgctgagcattatcaacaaacggaa

SEQ ID NO: 2 - DNA, 21 bp, synthetic construct
  misc_feature 1..21: note = DENV3_F3
  source 1..21: mol_type = other DNA
cttaacgtagtgctgacagtt

SEQ ID NO: 3 - DNA, 18 bp, synthetic construct
  misc_feature 1..18: note = DENV3_B3a
  source 1..18: mol_type = other DNA
ctgctgttggtggaatgg

SEQ ID NO: 4 - DNA, 48 bp, synthetic construct
  misc_feature 1..48: note = DENV3_FIPv4
  source 1..48: mol_type = other DNA
tctcacgcgtttcagcatattgagagagcagatctctgatgaacaacc

SEQ ID NO: 5 - DNA, 42 bp, synthetic construct
  misc_feature 1..42: note = DENV3_BIPv6
  source 1..42: mol_type = other DNA
tggcgaagagattctcaagaggactagaaatctgaggaaagc

SEQ ID NO: 6 - DNA, 20 bp, synthetic construct
  misc_feature 1..20: note = DENV3_LF
  source 1..20: mol_type = other DNA
ttcccgtctttttccgttgg

SEQ ID NO: 7 - DNA, 17 bp, synthetic construct
  misc_feature 1..17: note = DENV3_LBv1
  source 1..17: mol_type = other DNA
attggttatggcgttta

SEQ ID NO: 8 - DNA, 30 bp, synthetic construct
  misc_feature 1..30: note = DENV3_Qp
  source 1..30: mol_type = other DNA
ctgaacggccaaggaccaatgaaattggtt

SEQ ID NO: 9 - DNA, 13 bp, synthetic construct
  misc_feature 1..13: note = DENV3_B3-m12
  source 1..13: mol_type = other DNA
atctagccaagac

SEQ ID NO: 10 - DNA, 13 bp, synthetic construct
  misc_feature 1..13: note = DENV3_B3-m13
  source 1..13: mol_type = other DNA
ctagccaagactc

SEQ ID NO: 11 - DNA, 13 bp, synthetic construct
  misc_feature 1..13: note = DENV3_B3-m14
  source 1..13: mol_type = other DNA
agccaagactcct

SEQ ID NO: 12 - DNA, 13 bp, synthetic construct
  misc_feature 1..13: note = DENV3_B3-m16
  source 1..13: mol_type = other DNA
gacttcttgaagg

SEQ ID NO: 13 - DNA, 13 bp, synthetic construct
  misc_feature 1..13: note = DENV3_B3-m18
  source 1..13: mol_type = other DNA
tcttgaaggttcc

SEQ ID NO: 14 - DNA, 13 bp, synthetic construct
  misc_feature 1..13: note = DENV3_B3-m19
  source 1..13: mol_type = other DNA
ttgaaggttcccc

SEQ ID NO: 15 - DNA, 13 bp, synthetic construct
  misc_feature 1..13: note = DENV3_B3-m20
  source 1..13: mol_type = other DNA
ttcttgaaggttc

SEQ ID NO: 16 - DNA, 13 bp, synthetic construct
  misc_feature 1..13: note = DENV3_B3-m21
  source 1..13: mol_type = other DNA
cttgaaggttccc

SEQ ID NO: 17 - DNA, 13 bp, synthetic construct
  misc_feature 1..13: note = DENV3_B3-m23
  source 1..13: mol_type = other DNA
aaggttccccatc

SEQ ID NO: 18 - DNA, 13 bp, synthetic construct
  misc_feature 1..13: note = DENV3_B3-m25
  source 1..13: mol_type = other DNA
ttgaaggttcccc

SEQ ID NO: 19 - DNA, 13 bp, synthetic construct
  misc_feature 1..13: note = DENV3_B3-m26
  source 1..13: mol_type = other DNA
gaaggttccccat

SEQ ID NO: 20 - DNA, 13 bp, synthetic construct
  misc_feature 1..13: note = DENV3_B3-m27
  source 1..13: mol_type = other DNA
aggttccccatct

SEQ ID NO: 21 - DNA, 13 bp, synthetic construct
  misc_feature 1..13: note = DENV3_F4-1
  source 1..13: mol_type = other DNA
gactcggaagctt

SEQ ID NO: 22 - DNA, 13 bp, synthetic construct
  misc_feature 1..13: note = DENV3_F4-2
  source 1..13: mol_type = other DNA
actcggaagcttg

SEQ ID NO: 23 - DNA, 14 bp, synthetic construct
  misc_feature 1..14: note = DENV3_F4-3
  source 1..14: mol_type = other DNA
gactcggaagcttg

SEQ ID NO: 24 - DNA, 12 bp, synthetic construct
  misc_feature 1..12: note = DENV3_F4-4
  source 1..12: mol_type = other DNA
gactcggaagct

SEQ ID NO: 25 - DNA, 12 bp, synthetic construct
  misc_feature 1..12: note = DENV3_F4-5
  source 1..12: mol_type = other DNA
actcggaagctt

SEQ ID NO: 26 - DNA, 12 bp, synthetic construct
  misc_feature 1..12: note = DENV3_F4-6
  source 1..12: mol_type = other DNA
ctcggaagcttg

SEQ ID NO: 27 - RNA, 1620 nt, synthetic construct
  misc_feature 1..1620: note = Hemagglutinin of Avian Influenza Virus (H5N1)
  source 1..1620: mol_type = other RNA
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagaaacacaacgggaagctctgcgatctagatggagtgaagcctctaattttgagagattgtagcgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattaggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaagccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccattggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaacaacttagaaaggagaatagagaatttaaacaagaagatggaagacgggttcctagatgtctggacttataatgctgaacttctggttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaatggaacgtatgactacccgcagtattcagaagaagcgagactaaaaagagaggaaataagtggagtaaaattggaatcaataggaatttaccaaatactgtcaatttattctacagtggcg

SEQ ID NO: 28 - DNA, 19 bp, synthetic construct
  misc_feature 1..19: note = H5N1-F3v7
  source 1..19: mol_type = other DNA
caattttaaagccgaatga

SEQ ID NO: 29 - DNA, 17 bp, synthetic construct
  misc_feature 1..17: note = H5N1-B3
  source 1..17: mol_type = other DNA
gtcgcaaggactaatct

SEQ ID NO: 30 - DNA, 47 bp, synthetic construct
  misc_feature 1..47: note = H5N1-FIP
  source 1..47: mol_type = other DNA
accatattccaactcacttttcataatttcattgcaccagaatatgc

SEQ ID NO: 31 - DNA, 34 bp, synthetic construct
  misc_feature 1..34: note = H5N1-BIP
  source 1..34: mol_type = other DNA
caaactccaatgggggcatggtgagagggtgtat

SEQ ID NO: 32 - DNA, 20 bp, synthetic construct
  misc_feature 1..20: note = H5N1-LF
  source 1..20: mol_type = other DNA
gagtcccctttcttgacaat

SEQ ID NO: 33 - DNA, 19 bp, synthetic construct
  misc_feature 1..19: note = H5N1-LB
  source 1..19: mol_type = other DNA
gataaactctagtatgcca

SEQ ID NO: 34 - DNA, 12 bp, synthetic construct
  misc_feature 1..12: note = H5N1-F4v2
  source 1..12: mol_type = other DNA
aaagtggaagga

SEQ ID NO: 35 - DNA, 12 bp, synthetic construct
  misc_feature 1..12: note = H5N1-F4v6
  source 1..12: mol_type = other DNA
gggcaaagtgga

SEQ ID NO: 36 - DNA, 12 bp, synthetic construct
  misc_feature 1..12: note = H5N1-F4v7
  source 1..12: mol_type = other DNA
cgggcaaagtgg

SEQ ID NO: 37 - DNA, 13 bp, synthetic construct
  misc_feature 1..13: note = H5N1-F4v17
  source 1..13: mol_type = other DNA
gattggtaccaag

SEQ ID NO: 38 - DNA, 12 bp, synthetic construct
  misc_feature 1..12: note = H5N1-B4v1
  source 1..12: mol_type = other DNA
tttctgagccca

SEQ ID NO: 39 - DNA, 12 bp, synthetic construct
  misc_feature 1..12: note = H5N1-B4v6
  source 1..12: mol_type = other DNA
ggctatttctga

SEQ ID NO: 40 - DNA, 12 bp, synthetic construct
  misc_feature 1..12: note = H5N1-B4v8
  source 1..12: mol_type = other DNA
agggctatttct

SEQ ID NO: 41 - RNA, 1530 nt, synthetic construct
  misc_feature 1..1530: note = NS5 of ZIKV
  source 1..1530: mol_type = other RNA
gtaatacgactcactatagggatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgactaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctg

SEQ ID NO: 42 - DNA, 18 bp, synthetic construct
  misc_feature 1..18: note = ZIKV F3
  source 1..18: mol_type = other DNA
cagaagggacctccgact

SEQ ID NO: 43 - DNA, 20 bp, synthetic construct
  misc_feature 1..20: note = ZIKV B3
  source 1..20: mol_type = other DNA
cgtaactggggtcttgtctt

SEQ ID NO: 44 - DNA, 42 bp, synthetic construct
  misc_feature 1..42: note = ZIKV FIP
  source 1..42: mol_type = other DNA
ttgaccaggtagttctcccagtatggccaatgccatttgttc

SEQ ID NO: 45 - DNA, 42 bp, synthetic construct
  misc_feature 1..42: note = ZIKV BIP
  source 1..42: mol_type = other DNA
agggagaatggatgaccactgactcctcaatccacactctgt

SEQ ID NO: 46 - DNA, 18 bp, synthetic construct
  misc_feature 1..18: note = ZIKV LF
  source 1..18: mol_type = other DNA
cccagtcaactggcacag

SEQ ID NO: 47 - DNA, 15 bp, synthetic construct
  misc_feature 1..15: note = ZIKV LB
  source 1..15: mol_type = other DNA
catgcttgtggtgtg

SEQ ID NO: 48 - DNA, 29 bp, synthetic construct
  misc_feature 1..29: note = ZIKV Qp
  source 1..29: mol_type = other DNA
tggaacccagtcaactggcacagatgaac

SEQ ID NO: 49 - DNA, 14 bp, synthetic construct
  misc_feature 1..14: note = ZIKV_B4-3
  source 1..14: mol_type = other DNA
tagggaatgtctgt

SEQ ID NO: 50 - DNA, 12 bp, synthetic construct
  misc_feature 1..12: note = ZIKV_B4-13
  source 1..12: mol_type = other DNA
gaatgtctgtcc

SEQ ID NO: 51 - DNA, 13 bp, synthetic construct
  misc_feature 1..13: note = ZIKV_B4-14
  source 1..13: mol_type = other DNA
tgtctgtccattt

SEQ ID NO: 52 - DNA, 13 bp, synthetic construct
  misc_feature 1..13: note = ZIKV_F4-2
  source 1..13: mol_type = other DNA
ctcctttatttcc

SEQ ID NO: 53 - DNA, 17 bp, synthetic construct
  misc_feature 1..17: note = ZIKV_F4-3
  source 1..17: mol_type = other DNA
aaatcatatgcgcaaat

SEQ ID NO: 54 - RNA, 1374 nt, synthetic construct
  misc_feature 1..1374: note = N gene of RABV
  source 1..1374: mol_type = other RNA
atggatgccgacaagattgtgttcaaagtcaataatcaggtggtctctttgaagcctgagattatcgtggatcaatatgagtacaagtaccctgccatcaaggatttgaaaaagccttgtatcaccctagggaaagcccccgacttgaacaaagcatacaaatcagttttatcaggcatgaatgccgccaaacttgatccggatgatgtatgctcctacttggcagcagcaatgcagttctttgaggggacatgtccggaagactggaccagctatggaatcctgattgcacgaaaaggagacaggatcaccccaaactctctagtggagataaagcgtactgatgtagacgggaattgggctctgacaggaggcatggaattgacaagggaccccactgtctctgaacatgcatctttagtcggtcttctcctgagtctgtacaggttgagcaaaatatcaggacagaacactggtaactataagacaaacattgcagataggatagagcagattttcgagacagcaccttttgttaagatcgtggaacaccataccctaatgacaactcacaagatgtgtgctaattggagtactataccgaacttcagatttttggccggaacctacgacatgtttttctcacggattgagcatctgtattcggcaatcagagtgggcacagtcgtcaccgcttatgaagactgctcaggactggtatcgtttacagggttcataaagcagatcaatctcaccgcaagggaagcaatactatatttcttccacaagaactttgaggaagagataagaagaatgttcgagccagggcaagagacagctgttcctcactcttatttcatccacttccgttcactaggcttgagtgggaagtctccttattcatcgaatgctgtcggtcatgtgttcaatctcattcactttgttggatgctacatgggtcaagtcagatctctaaatgcgacggttattgctgcatgtgcccctcatgagatgtctgttctagggggctatttgggagaggaattcttcggaaaagggacatttgaaagaaggttcttcagagacgagaaagaacttcaagaatatgaggcggctgaactaacaaagaccgacgtggcactggcagatgacggaaccgtcaactctgatgacgaggactatttctccggtgaaaccagaagtccagaagctgtctatactcgaatcatgatgaatggaggtcgactgaagagatctcatatacggagatatgtctcagtcagttccaatcatcaagcccgtccaaactcattcgccgaatttttaaacaagacgtattcgaatgactcataaccctatagtgagtcgtattac

SEQ ID NO: 55 - DNA, 16 bp, synthetic construct
  misc_feature 1..16: note = RABV1-F3
  source 1..16: mol_type = other DNA
acatgtccggaagact

SEQ ID NO: 56 - DNA, 18 bp, synthetic construct
  misc_feature 1..18: note = RABV1-B3
  source 1..18: mol_type = other DNA
cagactcaggagaagacc

SEQ ID NO: 57 - DNA, 37 bp, synthetic construct
  misc_feature 1..37: note = RABV1-FIP
  source 1..37: mol_type = other DNA
actagagagtttggggtgaggaccagctatggaatcc

SEQ ID NO: 58 - DNA, 37 bp, synthetic construct
  misc_feature 1..37: note = RABV1-BIP
  source 1..37: mol_type = other DNA
acgggaattgggctctgacctaaagatgcatgttcag

SEQ ID NO: 59 - DNA, 22 bp, synthetic construct
  misc_feature 1..22: note = RABV1-LB
  source 1..22: mol_type = other DNA
ggcatggaattgacaagggacc

SEQ ID NO: 60 - DNA, 13 bp, synthetic construct
  misc_feature 1..13: note = RABV1-F4v14
  source 1..13: mol_type = other DNA
cagcagcaatgca

SEQ ID NO: 61 - DNA, 14 bp, synthetic construct
  misc_feature 1..14: note = RABV1-B4v3
  source 1..14: mol_type = other DNA
tattttgctcaacc

SEQ ID NO: 62 - DNA, 14 bp, synthetic construct
  misc_feature 1..14: note = RABV1-B4v7
  source 1..14: mol_type = other DNA
tgtcctgatatttt

(19) United States
(12) Patent Application Publication
(10) Pub. No.: US 2026/0275431 A1
(43) Pub. Date: Sep. 17, 2026

(54) KIT FOR DETECTING A SINGLE-STRANDED RNA VIRUS

(71) Applicant: EIKEN KAGAKU KABUSHIKI KAISHA, Tokyo (JP)

(72) Inventors: Machiko YAMAMOTO, Shimotsuga-gun (JP); Kota YOKONO, Shimotsuga-gun (JP); Syouhei SEMBA, Shimotsuga-gun (JP); Satoru MICHIYUKI, Otawara-shi (JP)

(73) Assignee: EIKEN KAGAKU KABUSHIKI KAISHA, Tokyo (JP)

(21) Appl. No.: 19/684,510

(22) Filed: May 21, 2026

Related U.S. Application Data
(63) Continuation of application No. 17/904,897, filed on Feb. 24, 2021, now Pat. No. 12,662,710.

(30) Foreign Application Priority Data
Feb. 27, 2020 (JP) 2020-031780

(51) Int. Cl. C12Q 1/70; C12Q 1/6844

(52) U.S. Cl. CPC C12Q 1/701; C12Q 1/6844

(57) ABSTRACT

Provided is a kit for detecting a single-stranded RNA virus. The kit comprises a primer set comprising (i) an FIP primer; (ii) a BIP primer; (iii) an F3 primer; (iv) a B3 primer; and (v) one additional outer primer.

### CROSS-REFERENCE TO RELATED APPLICATIONS

This application is a continuation of U.S. patent application Ser. No. 17/904,897, filed on Aug. 24, 2022, which is a U.S. National Stage Application filed under 35 U.S.C. § 371 of International Patent Application No. PCT/JP2021/006907, filed on Feb. 24, 2021, which claims the benefit of priority to Japanese Patent Application No. 2020-031780, filed on Feb. 27, 2020. The entire disclosures of each of the above-identified applications are hereby incorporated by reference in their entireties.

A computer readable xml file, entitled “046124-01-7104-US-01_SequenceListing.xml,” created on May 13, 2026 with a file size of 83,453 bytes contains the sequence listing for this application and is hereby incorporated by reference in its entirety.

### TECHNICAL FIELD

The present invention relates to a kit for detecting a single-stranded RNA virus.

### BACKGROUND

Viruses are organisms that parasitize cells and viruses cannot be cultured alone, and thus, a genetic diagnosis is mainly used as a method for identifying causative viruses in a short period of time in a diagnosis of a viral infectious disease. In a genetic diagnosis of a virus, a nucleic acid sequence unique to the target virus is amplified through a nucleic acid amplification reaction and detected.

A loop-mediated isothermal amplification (LAMP) method is known as a kind of nucleic acid amplification method (WO 00/28082). In this method, two kinds of inner primers (an FIP primer and a BIP primer) and two kinds of outer primers (an F3 primer and a B3 primer) are designed based on six regions (F3, F2, F1, B1c, B2c, and B3c, or F3c, F2c, F1c, B1, B2, and B3) of a target nucleic acid, and the target nucleic acid is amplified at a constant temperature using these primers. In addition, in the LAMP method, it is known that the efficiency of an amplification reaction is improved using two kinds of loop primers (a loop primer F and a loop primer B) in addition to the above-described inner primers and outer primers (WO 02/24902).

Viruses are classified into double-stranded DNA viruses, single-stranded RNA viruses, and the like, depending on the type of a nucleic acid of a genome. Single-stranded RNA viruses are classified into (+) strand (plus-strand) viruses in which genes are read in the direction from the 5′ terminal to the 3′ terminal and (−) strand (minus-strand) viruses in which genes are read in the direction from the 3′ terminal to the 5′ terminal via a complementary strand. When amplifying nucleic acids of RNA viruses, a reverse transcription reaction for synthesizing cDNA from template RNA using a reverse transcriptase is required at the start of an amplification reaction. The LAMP method combined with a reverse transcription reaction is called a reverse transcription loop-mediated isothermal amplification (RT-LAMP) method (for example, WO 2018/042598).

### SUMMARY

According to the LAMP methods disclosed in the above WO publications, viruses can be detected with relatively high sensitivity. However, since there are many diseases, among viral diseases, that cause severe symptoms, a method for more reliably detecting viruses that cause such diseases is needed. Therefore, an object of the present invention is to detect a single-stranded RNA virus with high sensitivity.

As a result of extensive studies, the present inventors have found that a single-stranded RNA virus can be detected with higher sensitivity by performing an RT-LAMP reaction using specific additional outer primers in addition to the conventional six kinds of primers, thus leading to realization of the present invention. According to the new findings of the present inventors, the sensitivity of detection is not improved when an outer primer that anneals to a region on the 3′ terminal side of a nucleic acid complementary to a target RNA of a single-stranded RNA virus is added, while the sensitivity of detection is improved when an outer primer that anneals to a region on the 3′ terminal side of a target RNA of a single-stranded RNA virus is added.

The present invention relates to following [1] to [8].

[1] A method for detecting a single-stranded RNA virus in a sample, the method comprising:
bringing a primer set into contact with a sample to perform a reverse transcription loop-mediated isothermal amplification reaction,
wherein the primer set is designed based on a nucleotide sequence of a target RNA of a single-stranded RNA virus and a nucleotide sequence of a nucleic acid complementary to the target RNA,
wherein when the single-stranded RNA virus is a plus-strand single-stranded RNA virus, the target RNA comprises arbitrary regions F3, F2, F1, B1c, B2c, and B3c in this order in a direction from a 5′ terminal to a 3′ terminal, and the nucleic acid complementary to the target RNA comprises arbitrary regions F3c, F2c, F1c, B1, B2, and B3 in this order in a direction from a 3′ terminal to a 5′ terminal,
wherein when the single-stranded RNA virus is a minus-strand single-stranded RNA virus, the target RNA comprises the arbitrary regions F3c, F2c, F1c, B1, B2, and B3 in this order in a direction from the 3′ terminal to the 5′ terminal, and the nucleic acid complementary to the target RNA comprises the arbitrary regions F3, F2, F1, B1c, B2c, and B3c in this order in a direction from the 5′ terminal to the 3′ terminal,
wherein the primer set comprises following (i) to (v):
(i) an FIP primer having the same nucleotide sequence as the region F1c at a 5′ terminal and having the same nucleotide sequence as the region F2 at a 3′ terminal;
(ii) a BIP primer having the same nucleotide sequence as the region B1c at a 5′ terminal and having the same nucleotide sequence as the region B2 at a 3′ terminal;
(iii) an F3 primer which is an outer primer having the same nucleotide sequence as the region F3;
(iv) a B3 primer which is an outer primer having the same nucleotide sequence as the region B3; and
(v) one or more additional outer primers, each additional outer primer having the same nucleotide sequence as an arbitrary region present on the 5′ terminal side from the region B3 or the region F3 in the nucleic acid complementary to the target RNA, and
wherein uracil that may be present in the nucleotide sequences of the FIP primer, the BIP primer, the F3 primer, the B3 primer, and the one or more additional outer primers may be substituted with thymine.
   [2] A method for detecting a plus-strand single-stranded RNA virus in a sample, the method comprising:
bringing a primer set into contact with a sample to perform a reverse transcription loop-mediated isothermal amplification reaction,
wherein the primer set is designed based on a nucleotide sequence of a target RNA comprising arbitrary regions F3, F2, F1, B1c, B2c, and B3c in this order in a direction from a 5′ terminal to a 3′ terminal and a nucleotide sequence of a nucleic acid complementary to the target RNA comprising arbitrary regions F3c, F2c, F1c, B1, B2, and B3 in this order in a direction from a 3′ terminal to a 5′ terminal, and comprises following (i) to (v):
(i) an FIP primer having the same nucleotide sequence as the region F1c at a 5′ terminal and having the same nucleotide sequence as the region F2 at a 3′ terminal;
(ii) a BIP primer having the same nucleotide sequence as the region B1c at a 5′ terminal and having the same nucleotide sequence as the region B2 at a 3′ terminal;
(iii) an F3 primer which is an outer primer having the same nucleotide sequence as the region F3;
(iv) a B3 primer which is an outer primer having the same nucleotide sequence as the region B3; and
(v) one or more additional outer primers, each additional outer primer having the same nucleotide sequence as an arbitrary region present on the 5′ terminal side from the region B3 in the nucleic acid complementary to the target RNA, and
wherein uracil that may be present in the nucleotide sequences of the FIP primer, the BIP primer, the F3 primer, the B3 primer, and the one or more additional outer primers may be substituted with thymine.
   [3] A method for detecting a minus-strand single-stranded RNA virus in a sample, the method comprising:
bringing a primer set into contact with a sample to perform a reverse transcription loop-mediated isothermal amplification reaction,
wherein the primer set is designed based on a nucleotide sequence of a target RNA comprising arbitrary regions F3c, F2c, F1c, B1, B2, and B3 in this order in a direction from a 3′ terminal to a 5′ terminal and a nucleotide sequence of a nucleic acid complementary to the target RNA comprising arbitrary regions F3, F2, F1, B1c, B2c, and B3c in this order in a direction from the 5′ terminal to the 3′ terminal, and comprises following (i) to (v):
(i) an FIP primer having the same nucleotide sequence as the region F1c at a 5′ terminal and having the same nucleotide sequence as the region F2 at a 3′ terminal;
(ii) a BIP primer having the same nucleotide sequence as the region B1c at a 5′ terminal and having the same nucleotide sequence as the region B2 at a 3′ terminal;
(iii) an F3 primer which is an outer primer having the same nucleotide sequence as the region F3;
(iv) a B3 primer which is an outer primer having the same nucleotide sequence as the region B3; and
(v) one or more additional outer primers, each additional outer primer having the same nucleotide sequence as an arbitrary region present on the 5′ terminal side from the region F3 in the nucleic acid complementary to the target RNA, and
wherein uracil that may be present in the nucleotide sequences of the FIP primer, the BIP primer, the F3 primer, the B3 primer, and the one or more additional outer primers may be substituted with thymine.
   [4] The method according to any one of [1] to [3],
wherein the primer set further comprises:
(vi) a loop primer F having the same nucleotide sequence as an arbitrary region between the region F1c and the region F2c; and
(vii) a loop primer B having the same nucleotide sequence as an arbitrary region between the region B1c and the region B2c, and
wherein uracil that may be present in the nucleotide sequences of the loop primer F and the loop primer B may be substituted with thymine.
   [5] The method according to any one of [1] to [4], wherein a melting temperature of the one or more additional outer primers of (v) is 30° C. to 55° C.
   [6] The method according to any one of [1] to [5], wherein the primer set comprises the one additional outer primer of (v).
   [7] The method according to any one of [1] to [6], the method further comprising: detecting an amplification product of the reverse transcription loop-mediated isothermal amplification reaction.
   [8] A kit for detecting a single-stranded RNA virus, the kit comprising:
a primer set,
wherein the primer set is designed based on a nucleotide sequence of a target RNA of a single-stranded RNA virus and a nucleotide sequence of a nucleic acid complementary to the target RNA,
wherein when the single-stranded RNA virus is a plus-strand single-stranded RNA virus, the target RNA comprises arbitrary regions F3, F2, F1, B1c, B2c, and B3c in this order in a direction from a 5′ terminal to a 3′ terminal, and the nucleic acid complementary to the target RNA comprises arbitrary regions F3c, F2c, F1c, B1, B2, and B3 in this order in a direction from a 3′ terminal to a 5′ terminal,
wherein when the single-stranded RNA virus is a minus-strand single-stranded RNA virus, the target RNA comprises the arbitrary regions F3c, F2c, F1c, B1, B2, and B3 in this order in the direction from the 3′ terminal to the 5′ terminal, and the nucleic acid complementary to the target RNA comprises the arbitrary regions F3, F2, F1, B1c, B2c, and B3c in this order in the direction from the 5′ terminal to the 3′ terminal,
wherein the primer set comprises following (i) to (v):
(i) an FIP primer having the same nucleotide sequence as the region F1c at a 5′ terminal and having the same nucleotide sequence as the region F2 at a 3′ terminal;
(ii) a BIP primer having the same nucleotide sequence as the region B1c at a 5′ terminal and having the same nucleotide sequence as the region B2 at a 3′ terminal;
(iii) an F3 primer which is an outer primer having the same nucleotide sequence as the region F3;
(iv) a B3 primer which is an outer primer having the same nucleotide sequence as the region B3; and
(v) one or more additional outer primers, each additional outer primer having the same nucleotide sequence as an arbitrary region present on the 5′ terminal side from the region B3 or the region F3 in the nucleic acid complementary to the target RNA, and
wherein uracil that may be present in the nucleotide sequences of the FIP primer, the BIP primer, the F3 primer, the B3 primer, and the one or more additional outer primers may be substituted with thymine.

According to the present invention, a single-stranded RNA virus can be detected with high sensitivity.

### BRIEF DESCRIPTION OF THE DRAWINGS

FIG. 1 shows regions F3, F2, F1, B1c, B2c, B3c, and B4c in a target RNA of a (+)ssRNA virus, regions F3c, F2c, F1c, B1, B2, B3, and B4 in cDNA complementary to the target RNA, and a primer set (an FIP primer, a BIP primer, an F3 primer, a B3 primer, a B4 primer, a loop primer F, and a loop primer B) designed based on these nucleotide sequences.

FIG. 2 shows regions F4c, F3c, F2c, F1c, B1, B2, and B3 in a target RNA of a (−)ssRNA virus, regions F4, F3, F2, F1, B1c, B2c, and B3c in cDNA complementary to the target RNA, and a primer set (an FIP primer, a BIP primer, an F3 primer, a B3 primer, an F4 primer, a loop primer F, and a loop primer B) designed based on these nucleotide sequences.

### DETAILED DESCRIPTION

A method for detecting a single-stranded RNA virus in a sample according to an aspect of the present invention is a method which involves detecting nucleic acid amplification through an RT-LAMP method to detect a single-stranded RNA virus in a sample, and is characterized by the use of one or more additional outer primers that anneal to a region on a 3′ terminal side of a target RNA in addition to the conventional primers (that is, an FIP primer, a BIP primer, an F3 primer, a B3 primer, and optionally an arbitrary loop primer F and a loop primer B) as a primer set. More specifically, the method for detecting a single-stranded RNA virus in a sample according to one aspect of the present invention comprises bringing a primer set into contact with a sample to perform an RT-LAMP reaction, wherein the primer set comprises an FIP primer, a BIP primer, an F3 primer, a B3 primer, and one or more additional outer primers to be described below, and optionally a loop primer F and a loop primer B.

A single-stranded RNA virus to be detected may be a plus-strand single-stranded RNA ((+)ssRNA) virus or may be a minus-strand single-stranded RNA ((−)ssRNA) virus. The (+)ssRNA virus is not particularly limited, and may be, for example, dengue viruses such as dengue virus type 3 (DENV3), rubella virus, hepatitis C virus, norovirus, chikungunya virus, zika virus (ZIKV), coronaviruses, human immunodeficiency virus (HIV), or hepatitis A virus. The (−)ssRNA virus is not particularly limited, and may be, for example, influenza viruses, Sendai virus, measles virus, human metapneumovirus, or rabies virus (RABV).

The sample may be a sample collected from a subject suspected of being infected with a single-stranded RNA virus, and may be, for example, sputum, body fluids, feces, or tissue. The body fluids may be, for example, nasal mucus, saliva, blood, serum, plasma, cerebrospinal fluid, urine, semen, or amniotic fluid. In addition, the sample may be bronchoalveolar lavage fluid, nasal suction fluid, nasal lavage fluid, a nasal swab, a pharyngeal swab, or mouthwash. Alternatively, the sample may be cells used in an experiment such as an infection experiment, or a culture liquid thereof. The sample may also be the above samples that have been subjected to pretreatment such as separation, extraction, concentration, and purification.

Target RNA may be the whole or a part of single-stranded RNA possessed by a single-stranded RNA virus. The length of the target RNA is not particularly limited, and may be, for example, 200 to 30,000 bases, 200 to 1,000 bases, or 200 to 550 bases.

The terms “FIP primer”, “BIP primer”, “F3 primer”, “B3 primer”, “loop primer F”, and “loop primer B” in the present specification are synonymous with these terms in the conventional LAMP method, and are understood by those skilled in the art. In other words, primers other than the additional outer primers to be described below can be designed by those skilled in the art as appropriate, based on the disclosure of known literature.

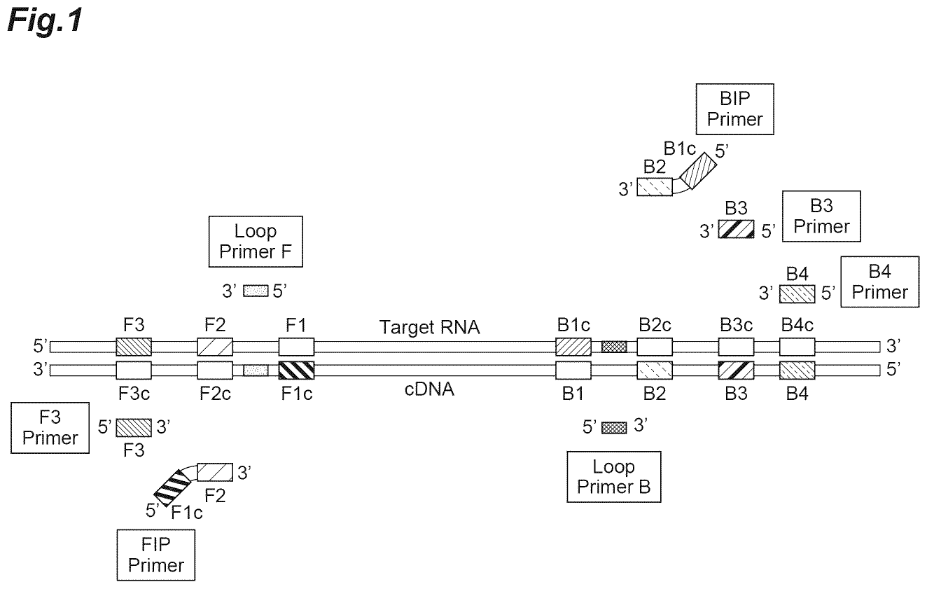

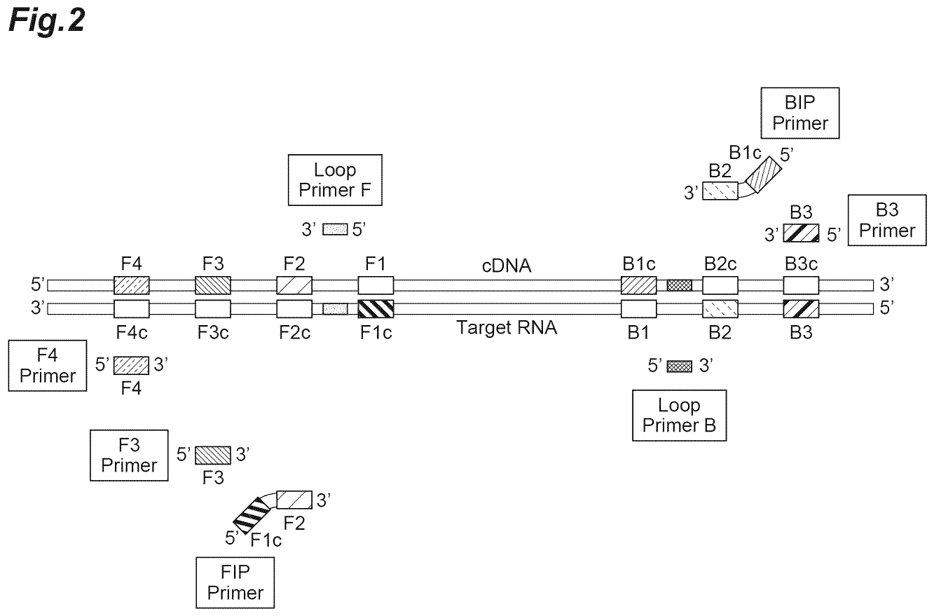

Hereinafter, a primer set of the method according to the present aspect will be described with reference to FIGS. 1 and 2. The primer set is designed based on a nucleotide sequence of target RNA of a single-stranded RNA virus and a nucleotide sequence of a nucleic acid complementary to the target RNA. Of the target RNA and the nucleic acid complementary to the target RNA, in a strand having a nucleotide sequence that can directly serve as a template for protein synthesis, arbitrary regions that serve as a base for a primer design are called regions F4, F3, F2, F1, B1c, B2c, B3c, and B4c in order from the 5′ terminal side to the 3′ terminal side. On the other hand, in a strand having a nucleotide sequence that cannot directly serve as a template for protein synthesis, arbitrary regions that serve as a base for a primer design are called regions F4c, F3c, F2c, F1c, B1, B2, B3, and B4 in order from the 3′ terminal side to the 5′ terminal side. The nucleotide sequences of the regions F4, F3, F2, F1, B1c, B2c, B3c, and B4c are respectively complementary to the regions F4c, F3c, F2c, F1c, B1, B2, B3, and B4. Accordingly, when a single-stranded RNA virus to be detected is a (+)ssRNA virus, the regions F3, F2, F1, B1c, B2c, B3c, and B4c are arbitrary regions present in this order in the direction from the 5′ terminal to the 3′ terminal of a target RNA, and the regions F3c, F2c, F1c, B1, B2, B3, and B4 are arbitrary regions present in this order in the direction from the 3′ terminal to the 5′ terminal of a nucleic acid complementary to the target RNA, as shown in FIG. 1. On the other hand, when a single-stranded RNA virus to be detected is a (−)ssRNA virus, the regions F4, F3, F2, F1, B1c, B2c, and B3c are arbitrary regions present in this order in the direction from the 5′ terminal to the 3′ terminal of a nucleic acid complementary to a target RNA, and the regions F4c, F3c, F2c, F1c, B1, B2, and B3 are arbitrary regions present in this order in the direction from the 3′ terminal to the 5′ terminal of the target RNA, as shown in FIG. 2. Although cDNA is shown in FIGS. 1 and 2 as a nucleic acid complementary to a target RNA, a nucleic acid complementary to a target RNA is not limited to DNA, and may be RNA.

In the present specification, the term “complementary” does not necessarily mean being completely complementary. For example, the scope of a “nucleotide sequence complementary to a certain nucleotide sequence” encompasses a nucleotide sequence of a nucleic acid that hybridizes with a nucleic acid having the certain nucleotide sequence under stringent conditions. The stringent conditions are not particularly limited but may be, for example, 50% formamide, 5×SSC (150 mL NaCl and 15 mM trisodium citrate), 50 mM sodium phosphate (pH 7.6), a 5×Denhardt's solution, and 10% dextran sulfate.

In the present specification, the term “same” does not necessarily mean being exactly the same. For example, the scope of the “same nucleotide sequence as a certain nucleotide sequence” encompasses a nucleotide sequence complementary to a nucleotide sequence complementary to the certain nucleotide sequence.

As is understood by those skilled in the art, the FIP primer, which is an inner primer, has the same nucleotide sequence as the region F1c at the 5′ terminal and the same nucleotide sequence as the region F2 at the 3′ terminal. However, when there is uracil in the region F1c or the region F2, the corresponding uracil in the FIP primer may be substituted with thymine. There may or may not be one or more nucleotides serving as a linker between the same nucleotide sequence as that of the region F1c and the same nucleotide sequence as that of the region F2. The nucleotides serving as a linker may be 1 to 500 bases, 1 to 100 bases, or 10 to 70 bases.

As is understood by those skilled in the art, the BIP primer, which is an inner primer, has the same nucleotide sequence as the region B1c at the 5′ terminal and the same nucleotide sequence as the region B2 at the 3′ terminal. However, when there is uracil in the region B1c or the region B2, the corresponding uracil in the BIP primer may be substituted with thymine.

As is understood by those skilled in the art, the F3 primer, which is an outer primer, has the same nucleotide sequence as the region F3. However, when there is uracil in the region F3, the corresponding uracil in the F3 primer may be substituted with thymine.

As is understood by those skilled in the art, the B3 primer, which is an outer primer, has the same nucleotide sequence as the region B3. However, when there is uracil in the region B3, the corresponding uracil in the B3 primer may be substituted with thymine.

As is understood by those skilled in the art, the loop primer F has the same nucleotide sequence as an arbitrary region between the region F1c and the region F2c. However, when there is uracil in the arbitrary region between the region F1c and the region F2c, the corresponding uracil in the loop primer F may be substituted with thymine.

As is understood by those skilled in the art, the loop primer B has the same nucleotide sequence as an arbitrary region between the region B1c and the region B2c. However, when there is uracil in the arbitrary region between the region B1c and the region B2c, the corresponding uracil in the loop primer B may be substituted with thymine.

As described above, those skilled in the art can appropriately design the FIP, BIP, F3, and B3 primers, the loop primer F, and the loop primer B suitable for amplification of the target RNA. In other words, those skilled in the art can appropriately select regions suitable as the regions F3, F2, F1, B1c, B2c, and B3c in the target RNA. The length (number of bases) of each region may be, for example, 5 bases or more, 10 bases or more, 5 to 200 bases, 10 to 25 bases, 10 to 20 bases, or 17 to 25 bases. The melting temperature (Tm) of each region may be, for example, 55° C. to 65° C., 60° C. to 65° C., or 55° C. to 60° C. The Tm value in the present specification is a value calculated by a nearest-neighbor method (In a reaction solution, sodium ion concentration: 50 mM, magnesium ion concentration: 4 mM, and oligonucleic acid concentration: 0.1 μM). The CG content in each region may be, for example, 40% to 60%, 40% to 50%, or 50% to 60%. The free energy change (dG) of 6 bases from the 5′ terminal of the regions F1c and B1c may be −4 kcal/mol or less. The dG of 6 bases from the 3′ terminal of the regions F2, B2, F3, B3 and the region serving as a base for designing loop primer F or B may also be −4 kcal/mol or less. Each primer preferably has a nucleotide sequence that does not form an extreme secondary structure. In addition, from the viewpoint of preventing formation of primer dimers, the 3′ terminal of each primer preferably does not have a nucleotide sequence complementary to its own 3′ terminal or the 3′ terminal of other primers.

The distance from the 5′ terminal of the region F2 to the 3′ terminal of the region B2c may be, for example, 120 to 180 bases. The distance between the region F2 and the region F3, and the distance between the region B2c and the region B3c may be, for example, 0 to 20 bases. The distance from the 5′ terminal of the region F2 to the 5′ terminal of the region F1, and the distance from the 5′ terminal of the region B2 to the 5′ terminal of the region B1 may be, for example, 40 to 60 bases. The distance from the region F1 to the region B1c is not particularly limited, and the 3′ terminal of the region F1 and the 5′ terminal of the region B1c may be directly connected to each other.

When a single-stranded RNA virus to be detected is a (+)ssRNA virus, the above-described additional outer primer has the same nucleotide sequence as an arbitrary region present on the 5′ terminal side from the region B3 in the nucleic acid complementary to the target RNA. However, when there is uracil in that region, the corresponding uracil in the above-described additional outer primer may be substituted with thymine. In FIG. 1, the B4 primer is the above-described additional outer primer, and the B4 primer has the same nucleotide sequence as an arbitrary region B4 present on the 5′ terminal side from the region B3 in cDNA complementary to the target RNA.

When a single-stranded RNA virus to be detected is a (−)ssRNA virus, the above-described additional outer primer has the same nucleotide sequence as an arbitrary region present on the 5′ terminal side from the region F3 in the nucleic acid complementary to the target RNA. However, when there is uracil in that region, the corresponding uracil in the above-described additional outer primer may be substituted with thymine. In FIG. 2, the F4 primer is the above-described additional outer primer, and the F4 primer has the same nucleotide sequence as an arbitrary region F4 present on the 5′ terminal side from the region F3 in cDNA complementary to the target RNA.

The Tm value of the above-described additional outer primer may be, for example, 30° C. to 55° C., 33° C. to 50° C., or 35° C. to 45° C. The length of the additional outer primer is not particularly limited, and may be, for example, 10 to 18 bases, 11 to 16 bases, or 12 to 14 bases. The CG content of the additional outer primer may be, for example, 30% to 70%, 34% to 65%, or 38% to 54%. The dG of 6 bases from the 3′ terminal of the additional outer primer may be −4 kcal/mol or less. The additional outer primer preferably has a nucleotide sequence that does not form an extreme secondary structure. In addition, from the viewpoint of preventing formation of primer dimers, the 3′ terminal of the additional outer primer preferably does not have a nucleotide sequence complementary to its own 3′ terminal or the 3′ terminal of other primers.

The location of the above-described arbitrary region (that is, the region B4 in FIG. 1 and the region F4 in FIG. 2) that serves as a base for designing the above-described additional outer primer is not particularly limited as long as a part or the whole of the region is present on the 5′ terminal side from the region B3 (in the case of the (+)ssRNA virus) or the region F3 (in the case of the (−)ssRNA virus) in the nucleic acid complementary to the target RNA, and the distance from the region B3 or the region F3 is not particularly limited. The distance from the 5′ terminal of the region B3 or the region F3 to the 3′ terminal of the above-described arbitrary region that serves as a base for designing the additional outer primer may be, for example, −4 to 50 bases, −3 to 35 bases, or −2 to 21 bases. Here, when the above-described arbitrary region that serves as a base for designing the outer primer partially overlaps with the region B3 or the region F3, the distance from the 5′ terminal of the region B3 or the region F3 to the 3′ terminal of the above-described arbitrary region that serves as a base for designing the additional outer primer is shown in a negative value.

Although the above-described additional outer primer is only the B4 primer in FIG. 1 and only the F4 primer in FIG. 2, the number of additional outer primers is not limited, and a plurality of additional outer primers may be included in a primer set. From the viewpoint of achieving higher detection sensitivity, the number of additional outer primers included in a primer set is preferably one.

Known reagents such as a reverse transcriptase, a DNA polymerase, and deoxynucleoside triphosphates (dNTPs: dATP, dTTP, dCTP, and dGTP) for performing an RT-LAMP reaction may be brought into contact with a sample together with the above-described primer set.

DNA polymerase is not particularly limited as long as it is a template-dependent nucleic acid synthase with a strand substitution activity, and may be, for example, a Bst DNA polymerase (large fragment), a Bca(exo−) DNA polymerase, a Csa DNA polymerase, Klenow fragment of Escherichia coli DNA polymerase I, or a combination thereof.

Reverse transcriptase is not particularly limited as long as it is an enzyme with an activity of synthesizing cDNA using RNA as a template, and may be, for example, a natural or recombinant reverse transcriptase derived from a natural or recombinant avian myeloblastosis virus (AMV), murine leukemia virus (MMLV), or human immunodeficiency virus (HIV). Examples of a reverse transcriptase derived from MMLV include SuperScript (registered trademark) II reverse transcriptase, SuperScript III reverse transcriptase, and SuperScript IV reverse transcriptase (all are manufactured by Thermo Fisher Scientific Inc.), and ReverTra Ace (registered trademark) (manufactured by TOYOBO Co., LTD.). Examples of a reverse transcriptase derived from AMV include ThermoScript (registered trademark) reverse transcriptase (manufactured by Thermo Fisher Scientific Inc.). Other specific examples of a reverse transcriptase include OmniScript (registered trademark) reverse transcriptase and Sensiscript (registered trademark) reverse transcriptase (both are manufactured by QIAGEN N.V.). When using an enzyme, such as a BCa(exo−) DNA polymerase, having both a reverse transcriptase activity and a DNA polymerase activity as a DNA polymerase, a separate reverse transcriptase may not be necessarily used.

Examples of other known reagents for performing an RT-LAMP reaction include a buffer solution or a salt that provides suitable conditions for enzymatic reactions, and a protective agent that stabilizes a template or an enzyme such as dithiothreitol (DTT).

A labeling probe or a fluorescent intercalator for detecting amplification products of an RT-LAMP reaction may be brought into contact with a sample together with the above-described primer set. The labeling probe may be, for example, a fluorescence labeling probe, and the fluorescence labeling probe may be, for example, a fluorescence quenching probe to be described below.

The step of bringing a primer set into contact with a sample to perform an RT-LAMP reaction may be performed, for example, by incubating a reaction solution containing the sample and the primer set.

The reaction solution may contain the above-described known reagents for performing an RT-LAMP reaction and the above-described labeling probe or fluorescent intercalator. The incubation time (that is, the reaction time of the RT-LAMP reaction) is usually 60 minutes or less, although it depends on the target RNA and the primer set. The incubation temperature (that is, the reaction temperature of the RT-LAMP reaction) is usually 65° C. or less.

The concentration of each inner primer in a reaction solution may be, for example, 0.8 to 2.4 μM. The concentration of each outer primer in a reaction solution may be, for example, 0.1 to 0.3 μM. The concentration of each loop primer in a reaction solution may be, for example, 0.4 to 1.2 μM. The concentration of an FIP primer may be, for example, 8 times or more of the concentration of an F3 primer and/or 1 to 4 times of the concentration of a loop primer F. The concentration of a BIP primer may be, for example, 8 times or more of the concentration of a B3 primer and/or 1 to 4 times of the concentration of a loop primer B.

In one embodiment, the method for detecting a single-stranded RNA virus in a sample may further comprise detecting an amplification product of an RT-LAMP reaction. When an amplification product of an RT-LAMP reaction is detected, it can be determined that there is a single-stranded RNA virus in the sample. The detection of an amplification product may be performed after the completion of the RT-LAMP reaction or may be performed in real time during the reaction.

The method for detecting an amplification product is not particularly limited, and known technology may be used. An amplification product may be detected, for example, by using a labeling probe, by using a fluorescent intercalator, or by performing electrophoresis on the reaction solution. Alternatively, in a case where magnesium ions are contained in the reaction solution, an amplification product may be detected by measuring the turbidity of the reaction solution. Magnesium ions react with pyrophosphate ions, which are by-products of nucleic acid synthesis, to produce white magnesium pyrophosphate.

As the labeling probe, a fluorescence labeling probe such as a fluorescence quenching probe (Quenching Probe: QProbe (registered trademark)), for example, may be used. Since fluorescence emitted from a fluorescence quenching probe is quenched when the probe is hybridized with a target nucleic acid, the amplification product can be quantified or detected by measuring the decrease in fluorescence. Examples of fluorescent labels that may be used in a fluorescence quenching probe include BODIPY (registered trademark), BODIPY-FL, carboxyrhodamine 6G (CR6G), carboxytetramethylrhodamine (TAMRA), Pacific Blue (registered trademark), and fluorescein-4-isothiocyanate (FITC).

As the fluorescent intercalator, known fluorescent intercalator such as SYTO (registered trademark) 63 Red Fluorescent Nucleic Acid Stain (manufactured by Thermo Fisher Scientific Inc.), for example, may be used.

Another aspect of the present invention is a kit for detecting a single-stranded RNA virus comprising the above-described primer set. The kit may further comprise the above-described known reagents such as a reverse transcriptase, a DNA polymerase, dNTPs, a buffer solution, a salt, and a protective agent for performing an RT-LAMP reaction. In addition, the kit may further comprise the above-described labeling probe or fluorescent intercalator.

### EXAMPLES

### [Test Example 1] Detection of (+)ssRNA Virus (DENV3)

### Reference Example 1

25 μL of a reaction solution having following composition was prepared in a 0.2 mL reagent tube:
20 mM Tricine (pH 8.6),
30 mM KCl,
8 mM MgSO₄,
1.4 mM dNTPs,
0.5% Tween 20,
1.6 mM DTT,
1.6 μM FIP Primer and BIP Primer,
0.2 μM F3 Primer and B3 Primer,
0.8 M Loop Primer F and Loop Primer B,
AMV Reverse Transcriptase 1.0 U (20 U/μL, Manufactured by Roche),
Bst DNA polymerase 22.8 U (Manufactured by New England Biolabs),
RNase Inhibitor (40 U/μL, Manufactured by Promega) 1 μL,
Template RNA (100 copies) 5 μL,
PPase 20 mU (Manufactured by New England Biolabs), and
0.04 μM QProbe (Registered Trademark)

As an FIP primer, a BIP primer, an F3 primer, a B3 primer, a Loop primer F, a loop primer B, and QProbe, DENV3_FIPv4, DENV3_BIPv6, DENV3_F3, DENV3_B3a, DENV3_LF, DENV3_LBv1, and DENV3_Qp shown in Table 1 were respectively used. Template RNA (SEQ ID NO: 1) was prepared by integrating cDNA prepared by RT-PCR from DENV3 Capsid gene into a plasmid, and transcribing and purifying RNA from the plasmid DNA. Script Max (registered trademark) Thermo T7 Transcription Kit (manufactured by TOYOBO Co., LTD., Code Number: TSK-101) was used for the transcription, and RNeasy (registered trademark) Mini Kit (manufactured by QIAGEN N.V., Catalog Number: No. 74104) was used for RNA purification. The 5′ terminal of QProbe was labeled with BODIPY, and the 3′ terminal thereof was phosphorylated.

TABLE 1

| Primer | SEQ | | Tm |
|---|---|---|---|
| or probe | ID NO. | Sequence (from 5′ to 3′) | (° C.) |
| DENV3_F3 | 2 | CTTAACGTAGTGCTGACAGTT | 57.41 |
| DENV3_B3a | 3 | CTGCTGTTGGTGGAATGG | 57.50 |
| DENV3_FIPv4 | 4 | TCTCACGCGTTTCAGCATATTG-A | 76.13 |
| | | GAGAGCAGATCTCTGATGAACAACC | |
| DENV3_BIPv6 | 5 | TGGCGAAGAGATTCTCAAGAGGA- | 73.41 |
| | | CTAGAAATCTGAGGAAAGC | |
| DENV3_LF | 6 | TTCCCGTCTTTTTCCGTTGG | 60.29 |
| DENV3_LBv1 | 7 | ATTGGTTATGGCGTTTA | 49.49 |
| DENV3_Qp | 8 | bodipy-CTGAACGGCCAAGGACC | 69.84 |
| | | AATGAAATTGGTT-PHO | |

An RT-LAMP reaction was performed (N=6) at 63° C. for 60 minutes using a real time quantitative PCR system LightCycler (registered trademark) 96 (manufactured by Roche). In addition, the same RT-LAMP reaction was performed (N=6) with the concentration of the B3 primer increased 2 to 4 times. An amplification product of the RT-LAMP reaction was detected by detecting quenching of QProbe in real time. The results are shown in Table 2. In addition, for reference, detection results in a case where the amount of template RNA was increased to 10,000 copies are concurrently shown in Table 2.

TABLE 2

| | | Concentration of B3 primer | | | |
|---|---|---|---|---|---|
| | | 0.2 μM | 0.4 μM | 0.6 μM | 0.8 μM |
| 10,000 | Detection time | 9.3 | 9.1 | 9.1 | 9.1 |
| Copies | (minute) | | | | |
| 100 Copies | Detection time | N.D. | N.D. | 22.3 | N.D. |
| | (minute) | N.D. | 13.3 | N.D. | 13.4 |
| | | 11.5 | 12.8 | N.D. | 12.2 |
| | | 14.2 | 12.9 | N.D. | 13.8 |
| | | 14.0 | 12.4 | 12.3 | 11.8 |
| | | N.D. | 18.6 | N.D. | 13.7 |
| | Average value | 13.2 | 14.0 | 17.3 | 13.0 |
| | SD | 1.5 | 2.6 | 7.1 | 0.9 |
| | CV (%) | 11.5 | 18.4 | 41.1 | 7.1 |
| NC | Detection time | N.D. | N.D. | N.D. | N.D. |
| | (minute) | | | | |

In the tables in the present specification, SD means a standard deviation, CV means a coefficient of variation, NC means negative control, and N.D. means no detection. In the present test example, cases where quenching of QProbe was not detected within 30 minutes were determined as no detection. As shown in Table 2, the detection sensitivity of the template RNA was not improved by increasing the concentration of the B3 primer.

### Example 1

An RT-LAMP reaction was performed (N=2) in the same manner as in Reference Example 1, except that a B4 primer was added to a reaction solution at a final concentration of 0.2 μM. As the B4 primer, the primers shown in Table 3 were used. For comparison, the same reaction was performed without a B4 primer. The results are shown in Table 4. In addition, for reference, detection results in a case where the amount of template RNA was increased to 10,000 copies are concurrently shown in Table 4.

TABLE 3

| | SEQ | Sequence | Tm |
|---|---|---|---|
| B4 Primer | ID NO. | (from 5′ to 3′) | (° C.) |
| DENV3_B3-m12 | 9 | ATCTAGCCAAGAC | 39.13 |
| DENV3_B3-m13 | 10 | CTAGCCAAGACTC | 40.44 |
| DENV3_B3-m14 | 11 | AGCCAAGACTCCT | 44.96 |
| DENV3_B3-m16 | 12 | GACTTCTTGAAGG | 37.89 |
| DENV3_B3-m18 | 13 | TCTTGAAGGTTCC | 39.79 |
| DENV3 B3-m19 | 14 | TTGAAGGTTCCCC | 43.56 |
| DENV3 B3-m20 | 15 | TTCTTGAAGGTTC | 36.95 |
| DENV3_B3-m21 | 16 | CTTGAAGGTTCCC | 41.53 |
| DENV3_B3-m23 | 17 | AAGGTTCCCCATC | 43.16 |
| DENV3_B3-m25 | 18 | TTGAAGGTTCCCC | 43.56 |
| DENV3_B3-m26 | 19 | GAAGGTTCCCCAT | 43.16 |
| DENV3_B3-m27 | 20 | AGGTTCCCCATCT | 44.34 |

TABLE 4

| | | B4 Primer | | | | | | |
|---|---|---|---|---|---|---|---|---|
| | | | B3- | B3- | B3- | B3- | B3- | B3- |
| | | None | m12 | m13 | m14 | m16 | m18 | m19 |
| 10,000 | Detection | 9.3 | 9.3 | 9.3 | 9.2 | 9.4 | 9.4 | 9.6 |
| Copies | time (min.) | | | | | | | |
| 100 | Detection | N.D. | 18.7 | 17.3 | 12.3 | 12.3 | 13.5 | 13.3 |
| Copies | time (min.) | 12.2 | 15.6 | 13.6 | 17.5 | 12.3 | 15.8 | 12.6 |
| | Average | 12.2 | 17.1 | 15.5 | 14.9 | 12.3 | 14.6 | 12.9 |
| | value | | | | | | | |
| | SD | — | 2.2 | 2.6 | 3.7 | 0.0 | 1.6 | 0.5 |
| | CV (%) | — | 12.6 | 16.9 | 24.8 | 0.2 | 10.9 | 3.8 |
| NC | Detection | N.D. | N.D. | N.D. | N.D. | N.D. | N.D. | N.D. |
| | time (min.) | | | | | | | |

| | | B4 Primer | | | | | |
|---|---|---|---|---|---|---|---|
| | | B3- | B3- | B3- | B3- | B3- | B3- |
| | | m20 | m21 | m23 | m25 | m26 | m27 |
| 10,000 | Detection | 9.3 | 9.4 | 9.4 | 9.3 | 9.5 | 9.5 |
| Copies | time (min.) | | | | | | |
| 100 | Detection | 17.2 | 12.8 | 15.6 | 13.6 | 13.8 | 13.1 |
| Copies | time (min.) | 13.8 | 15.5 | 12.2 | 14.7 | 14.7 | 13.2 |
| | Average | 15.5 | 14.2 | 13.9 | 14.1 | 14.3 | 13.1 |
| | value | | | | | | |
| | SD | 2.4 | 1.9 | 2.4 | 0.8 | 0.7 | 0.1 |
| | CV (%) | 15.6 | 13.4 | 17.4 | 5.8 | 4.7 | 0.8 |
| NC | Detection | N.D. | N.D. | N.D. | N.D. | N.D. | N.D. |
| | time (min.) | | | | | | |

As shown in Table 4, the detection sensitivity of the template RNA was improved by adding the B4 primer.

### Example 2

The same RT-LAMP reaction as that in Example 1 was performed with an increased number of times of measurement (N=6). As a B4 primer, B3-m27 was used. For comparison, the same reaction was performed without a B4 primer. The results are shown in Table 5. In addition, for reference, detection results in a case where the amount of template RNA was increased to 10,000 copies are concurrently shown in Table 5.

TABLE 5

| | | B4 Primer | |
|---|---|---|---|
| | | None | B3-m27 |
| 10,000 Copies | Detection time (minute) | 9.6 | 9.5 |
| 100 Copies | Detection time (minute) | N.D. | 13.8 |
| | | 12.6 | 14.3 |
| | | 13.0 | 14.3 |
| | | 13.7 | 11.4 |
| | | 15.1 | 27.4 |
| | | N.D. | 12.5 |
| | Average value | 13.6 | 15.6 |
| | SD | 1.1 | 5.9 |
| | CV (%) | 7.9 | 37.7 |
| NC | Detection time (minute) | N.D. | N.D. |

As shown in Table 5, the increased detection sensitivity due to the addition of the B4 primer was observed even when the number of times of measurement was increased.

### Comparative Example 1

The same RT-LAMP reaction as that in Example 1 was performed using F4 primers shown in Table 6 instead of the B4 primers. For comparison, the same reaction was performed without an F4 primer. The results are shown in Table 7. In addition, for reference, detection results in a case where the amount of template RNA was increased to 10,000 copies are concurrently shown in Table 7.

TABLE 6

| | SEQ | Sequence | Tm |
|---|---|---|---|
| F4 Primer | ID NO. | (from 5′ to 3′ ) | (° C.) |
| DENV3_F4-1 | 21 | GACTCGGAAGCTT | 44.07 |
| DENV3_F4-2 | 22 | ACTCGGAAGCTTG | 44.53 |
| DENV3_F4-3 | 23 | GACTCGGAAGCTTG | 47.26 |
| DENV3_F4-4 | 24 | GACTCGGAAGCT | 41.69 |
| DENV3_F4-5 | 25 | ACTCGGAAGCTT | 40.70 |
| DENV3 F4-6 | 26 | CTCGGAAGCTTG | 40.54 |

TABLE 7

| | | F4 Primer | | | | | | |
|---|---|---|---|---|---|---|---|---|
| | | None | F4-1 | F4-2 | F4-3 | F4-4 | F4-5 | F4-6 |
| 10,000 | Detection | 11.1 | 11.0 | 11.6 | 11.2 | 11.0 | 11.4 | 11.4 |
| Copies | time (minute) | | | | | | | |
| 100 | Detection | N.D. | 22.1 | 24.7 | 23.4 | N.D. | 26.3 | 18.9 |
| Copies | time (minute) | 19.3 | N.D. | 22.3 | 24.3 | 20.4 | 20.4 | 15.7 |
| | | 23.9 | 21.7 | 23.2 | N.D. | 19.6 | N.D. | 20.7 |
| | | N.D. | 18.4 | N.D. | N.D. | 25.4 | 18.0 | N.D. |
| | | N.D. | 18.7 | N.D. | N.D. | 15.1 | N.D. | 19.5 |
| | | N.D. | N.D. | N.D. | N.D. | N.D. | 18.7 | N.D. |
| | Average | 21.6 | 20.2 | 23.4 | 23.8 | 20.1 | 20.9 | 18.7 |
| | value | | | | | | | |
| | SD | 3.2 | 1.9 | 1.2 | 0.6 | 4.2 | 3.8 | 2.2 |
| | CV (%) | 14.9 | 9.6 | 5.2 | 2.6 | 20.9 | 18.0 | 11.5 |
| NC | Detection | N.D. | N.D. | N.D. | N.D. | N.D. | N.D. | N.D. |
| | time (minute) | | | | | | | |

As shown in Table 7, the detection sensitivity of the template RNA was not improved by adding the F4 primer.

### [Test Example 2] Detection of (−)ssRNA Virus (H5N1)

### Reference Example 2

25 μL of a reaction solution having following composition was prepared in a 0.2 mL reagent tube:
20 mM Tricine (pH 8.6),
30 mM KCl,
8 mM MgSO₄,
1.4 mM dNTPs,
0.5% Tween 20,
1.6 mM DTT,
1.6 μM FIP Primer and BIP Primer,
0.2 μM F3 Primer and B3 Primer,
0.8 μM Loop Primer F and Loop Primer B,
AMV Reverse Transcriptase 1.0 U (20 U/μL, Manufactured by Roche),
Bst DNA polymerase 22.8 U (Manufactured by New England Biolabs),
RNase Inhibitor (40 U/μL, Manufactured by Promega) 1 μL,
Template RNA (200 copies) 5 μL,
PPase 20 mU (Manufactured by New England Biolabs), and
0.1 μM SYTO 63 Red Fluorescent Nucleic Acid Stain (Manufactured by Thermo Fisher Scientific Inc.).

As an FIP primer, a BIP primer, an F3 primer, a B3 primer, a Loop primer F, and a loop primer B, H5N1-FIP, H5N1-BIP, H5N1-F3v7, H5N1-B3, H5N1-LF, and H5N1-LB shown in Table 8 were respectively used. Template RNA (SEQ ID NO: 27) was prepared by integrating cDNA prepared by RT-PCR from a part of Hemagglutinin (HA) gene of type H5 avian influenza virus (A/Viet Nam/1203/2004 (H5N1), Accession No. AY818135) in to a plasmid, and transcribing and purifying RNA from the plasmid DNA. Script Max Thermo T7 Transcription Kit (manufactured by TOYOBO Co., LTD., Code Number: TSK-101) was used for the transcription, and RNeasy Mini Kit (manufactured by QIAGEN N.V., Catalog Number: No. 74104) was used for RNA purification.

TABLE 8

| | SEQ | | Tm |
|---|---|---|---|
| Primer | ID NO. | Sequence (from 5′ to 3′) | (° C.) |
| H5N1-F3v7 | 28 | CAATTTTAAAGCCGAATGA | 50.82 |
| H5N1-B3 | 29 | GTCGCAAGGACTAATCT | 51.85 |
| H5N1-FIP | 30 | ACCATATTCCAACTCACTTTTCAT | 71.32 |
| | | AATT-TCATTGCACCAGAATATGC | |
| H5N1-BIP | 31 | CAAACTCCAATGGGGGC-ATGGTG | 74.29 |
| | | AGAGGGTGTAT | |
| H5N1-LF | 32 | GAGTCCCCTTTCTTGACAAT | 56.69 |
| H5N1-LB | 33 | GATAAACTCTAGTATGCCA | 49.94 |

An RT-LAMP reaction was performed (N=6) at 63° C. for 60 minutes using a real time quantitative PCR system LightCycler (registered trademark) 96 (manufactured by Roche). In addition, the same RT-LAMP reaction was performed (N=6) with the concentration of the F3 primer increased 2 to 4 times. An amplification product of the RT-LAMP reaction was detected by detecting fluorescence of an intercalator (SYTO 63 Red Fluorescent Nucleic Acid Stain) in real time. The results are shown in Table 9. In addition, for reference, detection results in a case where the amount of template RNA was increased to 10,000 copies are concurrently shown in Table 9.

TABLE 9

| | | Concentration of F3 primer | | | |
|---|---|---|---|---|---|
| | | 0.2 μM | 0.4 μM | 0.6 μM | 0.8 μM |
| 10,000 | Detection time | 7.3 | 7.2 | 7.4 | 7.2 |
| Copies | (minute) | | | | |
| 200 | Detection time | N.D. | 10.7 | N.D. | N.D. |
| Copies | (minute) | N.D. | N.D. | N.D. | N.D. |
| | | 10.7 | 12.6 | 9.7 | 16.8 |
| | | N.D. | N.D. | 27.4 | N.D. |
| | | 11.9 | N.D. | 9.9 | 10.8 |
| | | N.D. | 10.0 | N.D. | 11.7 |
| | Average value | 11.3 | 11.1 | 15.7 | 13.1 |
| | SD | 0.8 | 1.3 | 10.2 | 3.2 |
| | CV (%) | 7.5 | 11.8 | 65.0 | 24.7 |
| NC | Detection time | N.D. | N.D. | N.D. | N.D. |
| | (minute) | | | | |

In the present test example, in a case where the fluorescence of the intercalator had not been detected within 30 minutes, it was determined as not being detected. As shown in Table 9, the detection sensitivity of the template RNA was not improved by increasing the concentration of the F3 primer.

### Example 3

An RT-LAMP reaction was performed (N=6) in the same manner as in Reference Example 2, except that a F4 primer was added to a reaction solution at a final concentration of 0.2 μM. As the F4 primer, the primers shown in Table 10 were used. For comparison, the same reaction was performed without an F4 primer. The results are shown in Table 11. In addition, for reference, detection results in a case where the amount of template RNA was increased to 10,000 copies are concurrently shown in Table 11.

TABLE 10

| | SEQ | Sequence | Tm |
|---|---|---|---|
| F4 Primer | ID NO. | (from 5′ to 3′) | (° C.) |
| H5N1-F4v2 | 34 | AAAGTGGAAGGA | 35.84 |
| H5N1-F4v6 | 35 | GGGCAAAGTGGA | 42.81 |
| H5N1-F4v7 | 36 | CGGGCAAAGTGG | 45.50 |
| H5N1-F4v17 | 37 | GATTGGTACCAAG | 37.91 |

TABLE 1

| | | F4 Primer | | | | |
|---|---|---|---|---|---|---|
| | | None | F4v2 | F4v6 | F4v7 | F4v17 |
| 10,000 | Detection | 7.8 | 7.6 | 7.6 | 7.5 | 8.2 |
| Copies | time (minute) | | | | | |
| 200 | Detection | 10.8 | 10.0 | 10.3 | 9.1 | 11.3 |
| Copies | time (minute) | 17.6 | 11.5 | 10.2 | 11.4 | 13.0 |
| | | 15.8 | 13.1 | 9.7 | 11.3 | 13.1 |
| | | N.D. | 17.4 | 10.9 | 20.3 | 17.0 |
| | | 10.0 | 26.1 | 9.2 | 25.6 | 9.6 |
| | | N.D. | 11.9 | 11.4 | 17.6 | 11.0 |
| | Average | 13.5 | 15.0 | 10.3 | 15.9 | 12.5 |
| | value | | | | | |
| | SD | 3.7 | 6.0 | 0.8 | 6.4 | 2.5 |
| | CV (%) | 27.5 | 39.9 | 7.7 | 40.1 | 20.3 |
| NC | Detection | N.D. | N.D. | N.D. | N.D. | N.D. |
| | time (minute) | | | | | |

As shown in Table 11, the detection sensitivity of the template RNA was improved by adding the F4 primer.

### Comparative Example 2

The same RT-LAMP reaction as that in Example 3 was performed using B4 primers shown in Table 12 instead of the F4 primers. For comparison, the same reaction was performed without a B4 primer. The results are shown in Table 13. In addition, for reference, detection results in a case where the amount of template RNA was increased to 10,000 copies are concurrently shown in Table 13.

TABLE 12

| | SEQ | Sequence | Tm |
|---|---|---|---|
| B4 Primer | ID NO. | (from 5′ to 3′) | (° C.) |
| H5N1-B4v1 | 38 | TTTCTGAGCCCA | 40.36 |
| H5N1-B4v6 | 39 | GGCTATTTCTGA | 33.65 |
| H5N1-B4v8 | 40 | AGGGCTATTTCT | 34.94 |

TABLE 13

| | | B4 Primer | | | |
|---|---|---|---|---|---|
| | | None | B4v1 | B4v6 | B4v8 |
| 10,000 | Detection | 7.9 | 7.8 | 8.5 | 8.1 |
| Copies | time (minute) | | | | |
| 200 | Detection | 18.9 | 11.3 | 16.6 | 12.2 |
| Copies | time (minute) | 16.1 | N.D. | 18.0 | N.D. |
| | | N.D. | N.D. | 11.1 | N.D. |
| | | 20.9 | N.D. | N.D. | 11.0 |
| | | N.D. | N.D. | 17.5 | 10.7 |
| | | N.D. | N.D. | N.D. | 10.1 |
| | Average | 18.6 | 11.3 | 15.8 | 11.0 |
| | value | | | | |
| | SD | 2.4 | — | 3.2 | 0.9 |
| | CV (%) | 12.9 | — | 20.3 | 8.2 |
| NC | Detection | N.D. | N.D. | N.D. | N.D. |
| | time (minute) | | | | |

As shown in Table 13, the detection sensitivity of the template RNA was not improved by adding the B4 primer.

### [Test Example 3] Detection of (+)ssRNA Virus (ZIKV)

### Reference Example 3

The same RT-LAMP reaction as that in Reference Example 1 of Test Example 1 was performed, except that the template RNA and the primer set were changed as follows. As an FIP primer, a BIP primer, an F3 primer, a B3 primer, a Loop primer F, a loop primer B, and QProbe, ZIKV FIP, ZIKV BIP, ZIKV F3, ZIKV B3, ZIKV LF, ZIKV LB, and ZIKV Qp shown in Table 14 were respectively used. Template RNA (SEQ ID NO: 41) was prepared by integrating cDNA prepared by RT-PCR from NS5 gene of ZIKV into a plasmid, and transcribing and purifying RNA from the plasmid DNA. Script Max Thermo T7 Transcription Kit was used for the transcription, and RNeasy Mini Kit was used for RNA purification. The 3′ terminal of QProbe was labeled with BODIPY. The results are shown in Table 15.

TABLE 14

| Primer or | SEQ | Sequence | Tm |
|---|---|---|---|
| probe | ID NO. | (from 5′ to 3′) | (° C.) |
| ZIKV F3 | 42 | CAGAAGGGACCTCCGACT | 59.37 |
| ZIKV B3 | 43 | CGTAACTGGGGTCTTGTCTT | 58.84 |
| ZIKV FIP | 44 | TTGACCAGGTAGTTCTCCCAGT- | 75.65 |
| | | ATGGCCAATGCCATTTGTTC | |
| ZIKV BIP | 45 | AGGGAGAATGGATGACCACTGA- | 76.52 |
| | | CTCCTCAATCCACACTCTGT | |
| ZIKV LF | 46 | CCCAGTCAACTGGCACAG | 59.51 |
| ZIKV LB | 47 | CATGCTTGTGGTGTG | 50.08 |
| ZIKV Qp | 48 | TGGAACCCAGTCAACTGGCACA | 70.54 |
| | | GATGAAC-[BODIPY] | |

TABLE 15

| | | Concentration of B3 primer | | | |
|---|---|---|---|---|---|
| | | 0.2 μM | 0.4 μM | 0.6 μM | 0.8 μM |
| 10,000 | Detection time | 8.2 | 7.8 | 8.0 | 7.8 |
| Copies | (minute) | | | | |
| 100 Copies | Detection time | 15.7 | N.D. | 18.0 | 20.8 |
| | (minute) | 12.4 | N.D. | N.D. | 10.2 |
| | | N.D. | N.D. | N.D. | N.D. |
| | | 10.4 | N.D. | N.D. | 15.6 |
| | | 12.8 | 12.6 | N.D. | 13.2 |
| | | 21.7 | N.D. | N.D. | N.D. |
| | Average value | 14.6 | 12.6 | 18.0 | 15.0 |
| | SD | 4.4 | — | — | 4.5 |
| | CV (%) | 30.2 | — | — | 30.0 |
| NC | Detection time | N.D. | N.D. | N.D. | N.D. |
| | (minute) | | | | |

As shown in Table 15, the detection sensitivity of the template RNA was not improved by increasing the concentration of the B3 primer.

### Example 4

An RT-LAMP reaction was performed (N=6) in the same manner as in Reference Example 3, except that a B4 primer was added to a reaction solution at a final concentration of 0.2 μM. As the B4 primer, the primers shown in Table 16 were used. For comparison, the same reaction was performed without a B4 primer. The results are shown in Table 17. In addition, for reference, detection results in a case where the amount of template RNA was increased to 10,000 copies are concurrently shown in Table 17.

TABLE 16

| | SEQ | Sequence | Tm |
|---|---|---|---|
| B4 Primer | ID NO | (from 5′ to 3′) | (° C.) |
| ZIKV_B4-3 | 49 | TAGGGAATGTCTGT | 42.47 |
| ZIKV_B4-13 | 50 | GAATGTCTGTCC | 35.68 |
| ZIKV_B4-14 | 51 | TGTCTGTCCATTT | 39.05 |

TABLE 17

| | | B4 Primer | | | |
|---|---|---|---|---|---|
| | | None | B4-3 | B4-13 | B4-14 |
| 10,000 | Detection | 7.0 | 7.1 | 7.5 | 7.1 |
| Copies | time (minute) | | | | |
| 100 Copies | Detection | 23.7 | 13.0 | 12.0 | 19.2 |
| | time (minute) | N.D. | 12.3 | 11.0 | 11.7 |
| | | N.D. | 13.2 | 10.8 | 12.5 |
| | | 14.7 | 11.2 | 21.4 | 10.4 |
| | | N.D. | 20.0 | 22.4 | 16.8 |
| | | N.D. | 15.6 | 10.5 | 11.7 |
| | Average value | 19.2 | 14.2 | 14.7 | 13.7 |
| | SD | 6.4 | 3.2 | 5.6 | 3.5 |
| | CV (%) | 33.2 | 22.4 | 38.3 | 25.3 |
| NC | Detection | N.D. | N.D. | N.D. | N.D. |
| | time (minute) | | | | |

As shown in Table 17, the detection sensitivity of the template RNA was improved by adding the B4 primer.

### Comparative Example 3

The same RT-LAMP reaction as that in Example 4 was performed using F4 primers shown in Table 18 instead of the B4 primers. For comparison, the same reaction was performed without an F4 primer. The results are shown in Table 19. In addition, for reference, detection results in a case where the amount of template RNA was increased to 10,000 copies are concurrently shown in Table 19.

TABLE 18

| | SEQ | Sequence | Tm |
|---|---|---|---|
| F4 Primer | ID NO. | (from 5′ to 3′) | (° C.) |
| ZIKV_F4-2 | 52 | CTCCTTTATTTCC | 33.98 |
| ZIKV_F4-3 | 53 | AAATCATATGCGCAAAT | 48.21 |

TABLE 19

| | | F4 Primer | | |
|---|---|---|---|---|
| | | None | F4-2 | F4-3 |
| 10,000 | Detection time | 7.7 | 7.9 | 7.9 |
| Copies | (minute) | | | |
| 100 Copies | Detection time | 12.3 | N.D. | 13.1 |
| | (minute) | N.D. | N.D. | N.D. |
| | | 11.1 | 21.2 | N.D. |
| | | N.D. | N.D. | N.D. |
| | | N.D. | N.D. | N.D. |
| | | 10.5 | N.D. | N.D. |
| | Average value | 11.3 | 21.2 | 13.1 |
| | SD | 0.9 | — | — |
| | CV (%) | 8.1 | — | — |
| NC | Detection time | N.D. | N.D. | N.D. |
| | (minute) | | | |

As shown in Table 19, the detection sensitivity of the template RNA was not improved by adding the F4 primer.

### [Test Example 4] Detection of (−)ssRNA Virus (RABV)

### Reference Example 4

25 μL of a reaction solution having following composition was prepared in a 0.2 mL reagent tube:
20 mM Tricine (pH 8.6),
30 mM KCl,
8 mM MgSO₄,
1.4 mM dNTPs,
0.5% Tween 20,
1.6 mM DTT,
1.6 μM FIP Primer and BIP Primer,
0.2 μM F3 Primer and B3 Primer,
0.8 μM Loop Primer B,
AMV Reverse Transcriptase 1.0 U (20 U/μL, Manufactured by Roche),
Bst DNA polymerase 22.8 U (Manufactured by New England Biolabs),
RNase Inhibitor (40 U/μL, Manufactured by Promega) 1 μL,
Template RNA (10,000 copies) 5 μL,
PPase 20 mU (Manufactured by New England Biolabs), and
0.1 μM SYTO 63 Red Fluorescent Nucleic Acid Stain (Manufactured by Thermo Fisher Scientific Inc.).

As an FIP primer, a BIP primer, an F3 primer, a B3 primer, and a loop primer B, RABV1-FIP, RABV1-BIP, RABV1-F3, RABV1-B3, and RABV1-LB shown in Table 20 were respectively used. These primers are disclosed in Bazartseren B., S. Inoue, A. Yamada, et al. (2009): Rapid Detection of Rabies Virus by Reverse Transcription Loop-Mediated Isothermal Amplification. Jpn. J. Infect. Dis., 62, 187-191, 2009. Template RNA (SEQ ID NO: 54) was prepared by integrating cDNA prepared by RT-PCR from N gene of RABV into a plasmid, and transcribing and purifying RNA from the plasmid DNA. Script Max Thermo T7 Transcription Kit was used for the transcription, and RNeasy Mini Kit was used for RNA purification.

TABLE 20

| | SEQ | Sequence | Tm |
|---|---|---|---|
| Primer | ID NO. | (from 5′ to 3′) | (° C.) |
| RABV1-F3 | 55 | ACATGTCCGGAAGACT | 53.62 |
| RABV1-B3 | 56 | CAGACTCAGGAGAAGACC | 54.77 |
| RABV1-FIP | 57 | ACTAGAGAGTTTGGGGTGA- | 73.91 |
| | | GGACCAGCTATGGAATCC | |
| RABV1-BIP | 58 | ACGGGAATTGGGCTCTGAC- | 73.89 |
| | | CTAAAGATGCATGTTCAG | |
| RABV1-LB | 59 | GGCATGGAATTGACAAGGGACC | 63.58 |

An RT-LAMP reaction was performed (N=6) at 63° C. for 60 minutes using a real time quantitative PCR system LightCycler 96. In addition, the same RT-LAMP reaction was performed (N=6) with the concentration of the F3 primer increased 2 to 4 times. An amplification product of the RT-LAMP reaction was detected by detecting fluorescence of an intercalator (SYTO 63 Red Fluorescent Nucleic Acid Stain) in real time. The results are shown in Table 21. In addition, for reference, detection results in a case where the amount of template RNA was increased to 1×10⁶ copies are concurrently shown in Table 21.

TABLE 21

| | | Concentration of F3 primer | | | |
|---|---|---|---|---|---|
| | | 0.2 μM | 0.4 μM | 0.6 μM | 0.8 μM |
| 1 × 10⁶ | Detection time | 18.4 | 17.8 | 18.2 | 17.9 |
| Copies | (minute) | | | | |
| 10,000 | Detection time | N.D. | 23.2 | 24.2 | 27.4 |
| Copies | (minute) | 29.9 | 24.2 | 28.3 | N.D. |
| | | 28.9 | 25.6 | N.D. | 30.0 |
| | | 27.0 | N.D. | 25.6 | 25.3 |
| | | 29.4 | 27.8 | 27.7 | N.D. |
| | | N.D. | 25.3 | 28.1 | N.D. |
| | Average value | 28.8 | 25.2 | 26.8 | 27.6 |
| | SD | 1.3 | 1.7 | 1.8 | 2.3 |
| | CV (%) | 4.5 | 6.8 | 6.8 | 8.5 |
| NC | Detection time | N.D. | N.D. | N.D. | N.D. |
| | (minute) | | | | |

In the present test example, cases where the fluorescence of the intercalator was not detected within 30 minutes were determined as no detection. As shown in Table 21, the detection sensitivity of the template RNA was not improved by increasing the concentration of the F3 primer.

### Example 5

An RT-LAMP reaction was performed (N=6) in the same manner as in Reference Example 4, except that a F4 primer was added to a reaction solution at a final concentration of 0.2 μM. As the F4 primer, the primers shown in Table 22 were used. For comparison, the same reaction was performed without an F4 primer. The results are shown in Table 23. In addition, for reference, detection results in a case where the amount of template RNA was increased to 1×10⁶ copies are concurrently shown in Table 23.

TABLE 22

| | SEQ | Sequence | Tm |
|---|---|---|---|
| F4 Primer | ID NO. | (from 5′ to 3′) | (° C.) |
| RABV1-F4v14 | 60 | CAGCAGCAATGCA | 47.00 |

TABLE 23

| | | F4 Primer | |
|---|---|---|---|
| | | None | F4v14 |
| 1 × 10⁶ | Detection time | 17.3 | 17.4 |
| Copies | (minute) | | |
| 10,000 | Detection time | 24.8 | 26.6 |
| Copies | (minute) | 24.6 | 27.6 |
| | | N.D. | 25.3 |
| | | 28.1 | 26.7 |
| | | 25.1 | 27.7 |
| | | N.D. | 27.3 |
| | Average value | 25.7 | 26.8 |
| | SD | 1.7 | 0.9 |
| | CV (%) | 6.5 | 3.3 |
| NC | Detection time | N.D. | N.D. |
| | (minute) | | |

As shown in Table 23, the detection sensitivity of the template RNA was improved by adding the F4 primer.

### Comparative Example 4

The same RT-LAMP reaction as that in Example 5 was performed using B4 primers shown in Table 24 instead of the F4 primers. For comparison, the same reaction was performed without a B4 primer. The results are shown in Table 25. In addition, for reference, detection results in a case where the amount of template RNA was increased to 1×10⁶ are concurrently shown in Table 25.

TABLE 24

| | SEQ | Sequence | Tm |
|---|---|---|---|
| B4 Primer | ID NO. | (from 5′ to 3′) | (° C.) |
| RABV1-B4v3 | 61 | TATTTTGCTCAACC | 40.21 |
| RABV1-B4v7 | 62 | TGTCCTGATATTTT | 37.00 |

TABLE 25

| | | B4 Primer | | |
|---|---|---|---|---|
| | | None | B4v3 | B4v7 |
| 1 × 10⁶ Copies | Detection time | 21.2 | 20.4 | 19.8 |
| | (minute) | | | |
| 10,000 Copies | Detection time | N.D. | N.D. | N.D. |
| | (minute) | N.D. | N.D. | N.D. |
| | | 27.5 | N.D. | N.D. |
| | | N.D. | N.D. | N.D. |
| | | N.D. | 28.5 | N.D. |
| | | N.D. | N.D. | N.D. |
| | Average value | 27.5 | 28.5 | — |
| | SD | — | — | — |
| | CV (%) | — | — | — |
| NC | Detection time | N.D. | N.D. | N.D. |
| | (minute) | | | |

As shown in Table 25, the detection sensitivity of the template RNA was not improved by adding the B4 primer.

What is claimed is:

1. A kit for detecting a single-stranded RNA virus, the kit comprising:
a primer set,
wherein the primer set is designed based on a nucleotide sequence of a target RNA of a single-stranded RNA virus and a nucleotide sequence of a nucleic acid complementary to the target RNA,
wherein the primer set comprises following (i) to (v):
(i) an FIP primer having the same nucleotide sequence as the region F1c at a 5′ terminal and having the same nucleotide sequence as the region F2 at a 3′ terminal;
(ii) a BIP primer having the same nucleotide sequence as the region B1c at a 5′ terminal and having the same nucleotide sequence as the region B2 at a 3′ terminal;
(iii) an F3 primer which is an outer primer having the same nucleotide sequence as the region F3;
(iv) a B3 primer which is an outer primer having the same nucleotide sequence as the region B3; and
(v) one additional outer primer wherein either (a) or (b):
(a) the single-stranded RNA virus is the plus-strand single-stranded RNA virus, the target RNA comprises the arbitrary regions F3, F2, F1, B1c, B2c, B3c, and B4c in this order in the direction from the 5′ terminal to the 3′ terminal, wherein the nucleic acid complementary to the target RNA comprises the arbitrary regions F3c, F2c, F1c, B1, B2, B3, and B4 in this order in the direction from the 3′ terminal to the 5′ terminal, and the one additional outer primer has a nucleotide sequence present on the 5′ terminal side from the region B4 in the nucleic acid complementary to the target RNA; or
(b) the single-stranded RNA virus is the minus-strand single-stranded RNA virus, the target RNA comprises the arbitrary regions F4c, F3c, F2c, F1c, B1, B2, and B3 in this order in the direction from the 3′ terminal to the 5′ terminal, the nucleic acid complementary to the target RNA comprises the arbitrary regions F4, F3, F2, F1, B1c, B2c, and B3c in this order in the direction from the 5′ terminal to the 3′ terminal, and the one additional outer primer has a nucleotide sequence present on the 5′ terminal side from the region F4 in the nucleic acid complementary to the target RNA, and
wherein uracil that may be present in the nucleotide sequences of the FIP primer, the B1P primer, the F3 primer, the B3 primer, and the one additional outer primer may be substituted with thymine.

2. The kit according to claim 1,
wherein the primer set further comprises:
(vi) a loop primer F having the same nucleotide sequence as an arbitrary region between the region F1c and the region F2c; and
(vii) a loop primer B having the same nucleotide sequence as an arbitrary region between the region B1c and the region B2c, and
wherein uracil that may be present in the nucleotide sequences of the loop primer F and the loop primer B may be substituted with thymine.

3. The kit according to claim 1,
wherein a melting temperature of the one or more additional outer primer of (v) is 30° C. to 55° C.
